(19) United States
(12) Patent Application Publication
(10) Pub. No.: US 2012/0177619 A1
(43) Pub. Date: Jul. 12, 2012

(54) COMPOSITION COMPRISING A HAEMATIC COMPONENT AND ITS USE FOR THE TREATMENT OF DEGENERATIVE JOINT DISEASE

(76) Inventor: Valerio Di Nicola, Roma (IT)

(21) Appl. No.: 13/497,058

(22) PCT Filed: Sep. 22, 2010
(86) PCT No.: PCT/IB2010/054281

§ 371 Date: Mar. 20, 2012

(30) Foreign Application Priority Data
Sep. 23, 2009 (IT) RM2009A000485
Jun. 1, 2010 (IT) RM2010A000295

(51) Int. Cl. A61K 35/14; A61P 19/02

(52) U.S. Cl. 424/93.7; 424/529

(57) ABSTRACT

A composition including a haematic component, including blood cell components and heat shock protein, also including PDRN, alternatively including a thickener or mesenchymal stem cells, the process for preparing it, as well as its use for the treatment of degenerative joint disease.

### BACKGROUND OF THE INVENTION

The present invention refers to the field of pharmaceutics, in particular preparations able to be used in regenerative medicine.

Regenerative medicine is a recent field of research and clinical application of medicine based on cell therapy and tissue stimulation involving the new advancements in the fields of biology, medicine and biotechnology in a multidisciplinary fashion.

Therapeutic applications deriving from it have the goal of structurally and functionally maintaining, restoring and improving the organs and the apparatuses mainly dealing with degenerative diseases in the different phases of evolution (Parolini O., Soncini M. Human Placenta: a source of progenitor/stem cells? J. Reproduktionsmed. Endokrinol. 2006; 2:117-26).

Self-Repair is in particular a field of Regenerative medicine that deals with the organ-tissue repair mechanisms that can be activated and modulated in response when a disease, usually degenerative, causes them to progressively deteriorate.

More specifically, the present invention refers to the treatment of degenerative joint diseases (DJD).

Degenerative joint disease (DJD) progressively affects the cartilage that wears down until erosion and fragmentation. The articular capsule, the tendon and ligament structures thicken and stiffen due to the increased amount of collagen type III at the expense of type I-II, the increase in hyaline protein matrix and the decrease in the amount of elastin. The result is the loss of compliance and joint flexibility. Finally, the bone tissue erodes and deforms.

The pain is caused by the arthritic process, an inflammatory reaction that causes an increase in intra-articular pressure, which is also contributed to by the low compliance of the articular capsule; this promotes contact and friction between the articular surfaces accelerating cartilage erosion and subchondral inflammation.

The reduction in articular motility is thus caused by the onset of the inflammatory process and then by the degenerative involution of the articular constituents, until ankylosis.

Current therapeutic options in degenerative joint disease are steroid and non-steroid based anti-inflammatories for general and local use, analgesics, physical therapies like for example ultrasound, laser, hyperthermy, physiotherapy, intra-articular infiltration of hyaluronic acid and surgical therapy with arthroscopy or prosthetic surgery. Surgical treatment is preferred in the advanced forms of the disease.

From the second half of the nineteen sixties some Degenerative joint diseases (DJD) were treated using nonviable amniotic membranes, where by nonviable amniotic membranes we mean the amniotic membranes subjected to chemical-physical procedures that cause the destruction and removal of the cell component leaving as biologically active element just the subepithelial support layer, known as the commercial product Amniex®, (Mastelli s.r.l., Sanremo, Imperia, IT) i.e. a piece of human amniotic membrane rendered sterile, suspended in sodium chloride, sodium salicylate and distilled water preservative liquid.

In the 1980's Vishwakarma and Khare reported their experiences using amniotic nonviable membranes to restore articular tissues damaged by degenerative or post-infectious arthritis. They reported 25 patients, out of 28 underwent arthroplastics, were free of symptoms, with a good range of movement and stable joint. But the most important achievement was the structural regeneration of the articulation as follow-up showed during X-ray control (Vishwakarma G K, Kahare A K. Amniotic arthroplasty for tuberculosis of the hip. J. Bone Joint Surg. 1986; 68 (1): 68-74).

The nonviable amniotic membranes in contact with the damaged joints induced a clinical and structural improvement, which was radiologically evident (Vishwakarma G K, Kahare A K. Amniotic arthroplasty for tuberculosis of the hip. J. Bone Joint Surg. 1986; 68 (1): 68-74; Jin C Z, Park S R, Choi B H, Lee K Y, Kang O K, Min B H. Human amniotic membranes as a delivery matrix for articular cartilage repair. Tissue Eng. 2007; 13: 693-702). For about 15 years these non-viable amniotic membranes have no longer been on the market.

In 1910, Davis was the first describing the use of the fetal membrane as a surgical support material in skin transplantation at Johns Hopkins Hospital. Since then the use of amniotic membrane in surgery have expanded, from treatment of skin wounds to burn injuries, chronic leg ulcers, ocular surface disorders (corneal or conjunctiva disease) and prevention of post surgical adhesion formation.

At that time, research and therapy were directed towards substances with high biological potential empirically identified in the human placenta and in tissues subjected to prolonged stress until the premortal stage to release biological factors capable of activating a regenerative biological stimulus reaction (Davis I W. Skin transplantation with a review of 550 cases at the Johns Hopkins Hospital. Johns Hopkins Med. J. 1910; 15: 307-96).

Thereafter the biological regulators were identified as endorphins, growth-factors and locally-acting hormones and neuromediators.

The use of amniotic nonviable membranes treated under high temperature sterilization has been in use from the 1960's to the 1980's. In this period it did not contain viable cells and its biological properties were thus unidentified. Amnios so prepared was in therapeutic use for burn injuries and chronic leg ulcers as biological dressing (Trelford J D, Trelford-Sauder M. The amnion in surgery, past and present. Am. J. Obstet. Gynecol. 1979; 134; 833-46; Gruss J S, Jirsch D W. Human amniotic membrane: a versatile wound dressing. Can. Med. Assoc. J. 1978; 118: 1237-46; Ward D J, Bennet J P, Burgos H, Fabre J. The healing of chronic venous leg ulcers with prepared human amnion. Br. J. Plast. Surg. 1989; 42:463-7; Bilic G, Ochsenbein-Kolble N, Hall H, Huch R, Zimmerman R. In vitro lesion repair by human amnion epithelial and mesenchymal cells. Am. J. Obstet. Gynecol. 2004; 190: 87-92).

Filatov (1875-1956) investigated what happens to tissues before death or under significant biological stress. He used to keep fresh placenta tissue and amnios at very low temperatures for several days, before application. Filatov was convinced that, not only placenta tissue has repairing properties, but that any tissue put under extreme stress before dying had such properties. He thought that these biological activities were enhanced by “tissue suffering”, with the release of biohumoral substances that he named “bio-stimuline” (Editorial: Vladimir Petrovich Filatov. Brit. J. Ophtal. 1975; 59: 461; Filatov V P. Tissue Therapy. Khirurgiia (Mosk) 1955; 4: 3-6.)

Cerletti (1877-1963), a renowned neurologist and the “father of electroshock”, came to a similar conclusion after experimenting with electroshock and stress induction on pigs. In his experiments, Cerletti put pigs through high voltage electroshock, extracted their brain matter and injected it to another set of pigs. The second group of animals could tolerate the electro-stimulation much better than the control group. Moreover he noted that taking a sample of brain extracts obtained from pigs that underwent electroshock, and injecting it to pigs made comatose by electroshock treatment, he reached a temporary state of awakening.

The experimental evidences convinced Cerletti that a tissue put under extreme stress conditions enhances the production of substances which he called “acroagonine”, able to induce a powerful biological reaction (Kalinowsky L B. History of convulsive therapy. Ann. NY Acad. Sci. 1986; 462:1-4; Pallanti S. Images in Psychiatry: Ugo Cerletti 1877-1963. Am. J. Psychiatry 1999; 156: 630).

According to the present invention, by PDRN we mean polydeoxyribonucleotide, a linear polyanion, the monomer units of which are deoxyribonucleotides bonded by a phosphodiester bond with average molecular weight of about 350 KD (Tonello G, Daglio M, Zaccanelli N, Sottofattori E, Mazzei M, Balbi A, Characterization and quantification of the active polynucleotide (PDRN) from human placenta, a tissue repair stimulating agent, J. Pharm. Biomed. Anal. 1996, 14(11) 1555-60).

PDRN is also defined as a mixture of thermostable deoxyribonucleotides consisting of polymers of variable length between 50 and 2000 bases and of nucleosides obtained from salmon and trout sperm through a purification and sterilization process that ensures a high DNA concentration in the absence of peptides and proteins (Bianchini P, Tellini N, Morani A M, Follano M G. Pharmacological data on polydeoxyribonucleotide of human placenta; Guizzardi S, Galli C, Covoni et al. Polydeoxyribonucleotide (PDRN) promotes human osteoblast proliferation: A new proposal for bone tissue repair. Life Sciences 2003; 73:1973-83; Bianchini P, Tellini N, Morani A M, Follano M G. Pharmacological data on polydeoxyribonucleotide of human placenta. Int. J. of Tissue React. 1981; 3:151-54).

Initially, the wound repairing effects of PDRN has been associated with its property to induce the formation of platelet-fibronectin complexes (Bianchini P, Tellini N, Morani A M, Follano M G. Pharmacological data on polydeoxyribonucleotides of human placenta. Int. J. of Tissue React. 1981; 3:151-54; Surace A, Previtera A M, Mineo G, Barberis F. Valutazione dell'efficacia degli estratti placentari sulla sintomatologia algica dell'artrosi. Ortopedia e Traumatologia Oggi 1987; 7: 47-56).

PDRN is thermoresistant and therefore it can be subjected to heat treatment, for example in an autoclave at 121° C., obtaining the maximum guarantee of sterility.

PDRN is present on the market with the trade name Placentex® (Mastelli S.r.l., Sanremo, Imperia, IT) in a sodium chloride and distilled water preservative solution.

Placentex® (Mastelli S.r.l., Sanremo, Imperia, IT) has an effect in the repair and trophism of connective tissues that is obtained both with tropism towards the damaged outbreak, through the complex that is formed by affinity of PDRN towards platelets and fibronectin, and with stimulation for cell regeneration. This effect is attributed in part to the synergic action of PDRN and its metabolites with growth factors and in part to the activation of the metabolic salvage pathways that produce significant energy savings in the neo-synthesis of DNA, an essential step of tissue repair.

The current therapeutic indications for PDRN cover some chronic dermatological diseases having a dystrophic-degenerative character (dystrophic wounds, burns, ulcers of the lower limbs, anal fissures, post-actinic skin lesions) and ophthalmic character (conjunctival and corneal dystrophies) but, in literature, there is also experience of therapy on dystrophic lesions of the ectocervix (Pasquinucci C, Contini V. Computerized evaluation of reparative processes of the cervix uteri. Ann. Ostet. Ginecol. Med. Perinat. 1990; 111(6):364-71).

Recently it has been shown on an experimental model that there is a reparative property of PDRN on damaged endothelium and on the promotion of neoangiogenesis (Bitto A, Polito F, Altavilla D, Minutoli L, Migliorato A, Squadrito F. Polydeoxyribonucleotide (PDRN) restores blood flow in an experimental model of peripheral artery occlusive disease. J. Vasc. Surg. 2008; 48(5): 1292-300; Montesinos M C, Desai A, Chen J F et al. Adenosine promotes wound healing and mediates angiogenesis in response to tissue injury via occupancy of A2a receptors. Am. J. Pathol. 2002; 160: 2009-18).

PDRN was compared to FANS for the anti-inflammatory and antalgic effect on rheumatic pains, hypothesising for PDRN an anticomplementary effect and an effect promoting the suppression of the expression of cytokine (Surace A, Previtera A M, Mineo G, Barberis F. Valutazione dell'efficacia degli estratti placentari sulla sintomatologia algica dell'artrosi. Ortopedia e Traumatologia Oggi 1987; 7: 47-56).

Initially, the reparative effects of polydeoxyribonucleotides on skin wounds were associated with the property of promoting the formation of the platelet-fibronectin complex. It has recently been demonstrated how important the nucleosides and nucleotides present in the extracellular matrix are for stimulation in the proliferative sense that they have on cells of different tissues. The fragmentation of the nucleic acids, which follows the cellular lysis after they have died, produces an extracellular matrix of nucleosides and nucleotides that seem to induce a local stimulus for tissue regeneration (Born G V, Kratzer M A. Source and concentration of extracellular adenosine triphosphate during haemostasis in rats, rabbits and man. J. Physiol. 1984; 354: 419-29; Ding G J I, Ning-Na W, Heppel H. Extracellular ATP shows synergistic enhancement of DNA synthesis when combined with agents that are active in wound healing or as neurotransmitters. Biochem. Biophys. Res. Commun. 1990; 166: 251-58; Gailit J, Clarck A F. Wound repair in the context of extracellular matrix. Curr. Opin. Cell Biol. 1994; 6: 717-725; Rathbone M P, Christjanson L, De Forge S et al. Extracellular purine nucleosides stimulate cell division and morphogenesis: pathological and physiological implications. Med. Hypotheses 1992; 37: 232-240; Rathbone M P, De Forge S, De Luca B et al. Purinergic stimulation of cell division and differentiation: mechanism and pharmacological implications. Med. Hypotheses 1992; 37: 213-219).

Several researchers have recently shown the importance of extra cellular nucleotides and nucleosides activity, inducing proliferation and activity of cells in different tissue. Nucleic acids, nucleosides and nucleotides deriving from their fragmentation, physiologically diffuse into the extra cellular environment as a result of cell lyses following cellular death, possibly providing a local stimulus for tissue regeneration (Born G V, Kratzer M A. Source and concentration of extracellular adenosine triphosphate during haemostasis in rats, rabbits and man. J. Physiol. 1984; 354: 419-29; Ding G J I, Ning Na W, Heppel H. Extracellular ATP shows Synergistic enhancement of DNA synthesis when combined with agents that are active in wound healing or as neurotransmitters. Biochem Biophys Res. Commun. 1990; 166: 251-58; Gailit J, Clarck A F. Wound repair in the context of extracellular matrix. Curr. Opin. Cell Biol. 1994; 6: 717-725; Rathbone M P, Christjanson L, De Forge S et al. Extracellular purine nucleosides stimulate cell division and morphogenesis: pathological and physiological implications. Med. Hypotheses 1992; 37: 232-240; Rathbone M P, De Forge S, De luca B et al. Purinergic stimulation of cell division and differentiation: mechanism and pharmacological implications. Med Hypotheses 1992; 37: 213-219).

It has been demonstrated in vitro that fragmented nucleic acids promote the proliferation of fibroblasts, of osteoblasts and of endothelial cells (Chavan A J, Haley B E. Interaction of nucleotides with acid fibroblast growth factor (FGF-1). Biochemistry 1994; 33: 193-202; Bourque W T, Gross M, Hall B K. Expression of four growth-factors during fracture repair. Int. J. Dev. Biol. 1993; 37: 573-79; Bowler W B, Buckley K A, Gartland A, Hipskind R A, Bilbe G, Gallagher J A. Extracellular nucleotide signalling: a mechanism for integrating local and systemic responses in the activation of bone remodelling. Bone 2001; 28 (5): 507-12; Canalis E, Mc Carthy T, Centerella M. Growth factors and the regulation of bone remodeling. J. Clin. Invest. 1988; 81: 277-81; Joyce M E, Jingushi S, Bolander M E. Transforming growth factor beta in the regulation of fracture repair. Orthop. Clin. North Am. 1990; 21: 199-209; Joyce M E, Jingushi S, Scully S P, Bolander M E. Role of growth factors in the fracture healing. Prog. Clin. Biol. Res. 1991; 365: 391-416).

Similar results have been obtained in vivo in experimental models, using polydeoxyribonucleotides (Pdm) to stimulate healing in gamma radiation-induced lesions in mice (Henning U G G, Wang Q, Gee N H, Von Borstel R C. Protection and repair of g-radiation induced. lesions in mice with DNA or Deoxyribonucleosides treatments. Mutation Research 1996; 35: 25-30).

Some biochemical studies have shown that polydeoxyribonucleotide is the product of enzyme lysis of nucleic acids after cellular death. Nucleotides like ATP and nucleosides from enzyme cleavage of nucleic acids activate the salvage pathways stimulating the purinergic receptors P1-P2. The stimulation of the A2 receptors, sub-class of the purinergic receptors P1, by nucleosides, induces the proliferation of fibroblasts in culture (Thellung S, Florio T, Maragliano A, Cattarini G, Schettini G. Polydeoxyribonucleotides enhance the proliferation of human skin fibroblasts: involvement of A2 purinergic receptors subtypes. Life Sciences 1999; 64 (18): 1661-1674).

Sini and Nakamura have demonstrated in vitro the PDRN upward effects on fibroblasts and osteoblasts proliferation and collagen production, which promotes an increase of fibroblast growth of about 20% (Sini P, Denti A, Cattarini G, Daglio M, Tira M E, Balduini C. Effect of polydeoxyribonucleotides on human fibroblasts in primari culture. Cell Biochem Funct 1999; 17: 107-114; Nakamura E, Uezono Y, Narusawa K et al. ATP activates DNA synthesis by acting on P2X receptors in human osteoblasts like MG-63 cells. Am J Phisiol Cell Phisiol 2000; 279 (2): 510-519

Similarly, in vitro and in vivo studies have shown the role of A2 receptors, stimulated by nucleosides, in wound healing processes (Vega C, Desai A, Montesinos M C, Cronstein B N. Adenosine A2a receptor agonists promote more rapid wound healing than recombinant human platelet derived growth factor (Beclaplermin Gel). J. Inflammation 2002; 26 (1): 19-24).

It has been demonstrated that nucleic acids diffused in extra cellular environment act in synergy with different growth factors like PDGF, FGF, EGF, TGF-beta, cytokines, growth factors production and even influence immunologic responses (Ding G J I, Ning Na W, Heppel H. Extracellular ATP shows synergistic enhancement of DNA synthesis when combined with agents that are active in wound healing or as neurotransmitters. Biochem. Biophys. Res. Commun. 1990; 166: 251-58; Gailit J, Clarck A F. Wound repair in the context of extracellular matrix. Curr. Opin. Cell. Biol. 1994; 6: 717-725; Chavan A J, Haley B E. Interaction of nucleotides with acid fibroblast growth factor (FGF-1). Biochemistry 1994; 33: 193-202; Edington S M. DNA and RNA therapeutics: unsolved riddles. Bio Technology 1992; 10: 993-996; Adjej A A, Yamamoto S, Kulkarni A. Nucleic acid and/or their components: a possible role in immune function. J. Nutr. Sci. Vitaminol. 1995; 41: 1-16).

Some researchers studied the biological process that, in bone regeneration, provides an osteo-inductive signal for the mesenchymal host precursor cell, inducing proliferation and differentiation into active osteoblasts. The molecules mainly implicated in this biological process are the Bone Morphogenetic Proteins (BMP) and other Growth Factors like PDGF, FGF, IGF, and TGF-beta (Bourque W T, Gross M, Hall B K. Expression of four growth-factors during fracture repair. Int. J. Dev. Biol. 1993; 37: 573-79; Canalis E, Mc Carthy T, Centerella M. Growth factors and the regulation of bone remodeling. J. Clin. Invest. 1988; 81: 277-81; Joyce M E, Jingushi S, Bolander M E. Transforming growth factor beta in the regulation of fracture repair. Orthop. Clin. North Am. 1990; 21: 199-209; Lane J M, Tomin E, Bostrom MPG. Biosynthetic bone grafting. Clin. Orthop. Relat. Res. 1999; 367s: 107-117).

It has also in part been proven how extracellular nucleic acids act in synergy with other growth-factors like PDGF, FGF, EGF, TGF-beta, and cytokine influencing the local immunological response (Guizzardi S, Galli C, Covoni et al. Polydeoxyribonucleotide (PDRN) promotes human osteoblast proliferation: A new proposal for bone tissue repair. Life Sciences 2003; 73:1973-83).

The effects of PDRN in vitro on cultures of osteoblasts, the stimulation of salvage pathways and the activation of the A2 receptors have been analysed, showing an increase in growth of osteoblasts of 21% in cultures with PDRN with respect to controls (Guizzardi S, Galli C, Covoni et al. Polydeoxyribonucleotide (PDRN) promotes human osteoblast proliferation: A new proposal for bone tissue repair. Life Sciences 2003; 73:1973-83; Guizzardi S, Martini D, Bacchelli B, Valdatta B, Thione A, et al. Effects of heat deproteinate bone and polynucleotides on bone regeneration: An experimental study on rat, Micron 38, 2007, 722-728).

In Guizzardi S, Galli C, Covoni P et al. Polydeoxyribonucleotide (PDRN) promotes human osteoblast proliferation: A new proposal for bone tissue repair. Life Sciences 2003; 73:1973-83 the author had evaluated the in vitro effects of PDRN using human cell cultures of osteoblasts. After 6 days of development in culture, the osteoblasts exposed to PDRN showed an increase in growth of about 21%, with respect to the controls. This study suggested that PDRN could stimulate growth of osteoblasts and analyzed the biochemistry and the receptors implicated in said effect, also confirming what had already been demonstrated on fibroblasts (Chavan A J, Haley B E. Interaction of nucleotides with acid fibroblast growth factor (FGF-1). Biochemistry 1994; 33: 193-202; Bourque W T, Gross M, Hall B K. Expression of four growth-factors during fracture repair. Int. J. Dev. Biol. 1993; 37: 573-79; Bowler W B, Buckley K A, Gartland A, Hipskind R A, Bilbe G, Gallagher J A. Extracellular nucleotide signalling: a mechanism for integrating local and systemic responses in the activation of bone remodelling. Bone 2001; 28 (5): 507-12; Canalis E, Mc Carthy T, Centerella M. Growth factors and the regulation of bone remodeling. J. Clin. Invest. 1988; 81: 277-81; Joyce M E, Jingushi S, Bolander M E. Transforming growth factor beta in the regulation of fracture repair. Orthop. Clin. North Am. 1990; 21: 199-209; Joyce M E, Jingushi S, Scully S P, Bolander M E. Role of growth factors in the fracture healing. Prog. Clin. Biol. Res. 1991; 365: 391-416). It was highlighted that Bovine Serum (FBS) greatly influences cell growth in the presence of PDRN, attributing said effect to the presence in the serum of some active enzymes in the depolymerisation of PDRN that is decomposed into active nucleotides, capable of bonding and stimulating the purinergic receptors (P1-P2) (see also Nakamura E, Uezono Y, Narusawa K et al. ATP activates DNA synthesis by acting on P2X receptors in human osteoblasts like MG-63 cells. Am. J. Phisiol. Cell Phisiol. 2000; 279 (2): 510-519).

It is clear that in this context the fresh fetal bovine serum (FBS) was used as component for the cultivation of osteoblasts in vitro according to procedures known in the field.

In Guizzardi S, Martini D, Bacchelli B et al. Effects of heat deproteinate bone and polynucleotides on bone regeneration: An experimental study on rat. Micron 2007; 38: 722-28 the effect of PDRN on bone regeneration in rat was evaluated.

The experiment consisted of producing “holes” on the tibia cortical bone of the rat creating defects of substance to be filled with a paste consisting of deproteinate and triturated bone, with added PDRN. The elimination of the protein portion of the bone occurred through high temperature treatment. This matrix positioned in the holes of the rat bone induced a significantly greater bone regeneration with respect to the controls. It was not foreseen to use serum and/or other haematic derivatives.

Guizzardi et al., 2003 and Guizzardi et al., 2007, supra, do not disclose the treatment of Degenerative Joint Disease, which involves connective tissues of the joint capsule, such as cartilage ligaments and tendons.

In Guizzardi et al., 2003 PDRN is present together with PBS in the culture medium, since the first is voluntary added and the latter is present as a conventional additive for cell culturing, PDRN and PBS do not undergo to any specific treatment.

Therefore, it would be clear from the following detailed description that the present invention differs from the Guizzardi et al., 2003 and Guizzardi et al., 2007, supra, for two main reasons: firstly, the aim of the present invention is the treatment of Degenerative Joint Disease; furthermore the haematic component object of the present invention is obtained by thermally treating blood or a blood derivative, the latter containing blood cells.

International application WO94/15621 describes the use of oligodeoxyribonucleotides, polydeoxyribonucleotides and their derivatives for the treatment of various diseases like vascular thrombosis and HIV.

Hereafter we show the preliminary studies on the use of PDRN Placentex® (Mastelli S.r.l., Sanremo, Imperia, IT) in the treatment of degenerative joint diseases (DJD).

The preliminary clinical data is in contrast with what is known in the field on inducing the proliferation of fibroblasts.

The man skilled in the art is aware that the in vitro results must be validated by clinical evidence since the in vivo effect of the active ingredient depends upon its pharmacokinetics and its pharmacodynamics.

The inventors have surprisingly identified why PDRN is active in vitro but is ineffective in clinical practice.

Placentex® (Mastelli S.r.l., Sanremo, Imperia, IT) (5.625 mg) had already been tested for local infiltration use to evaluate its effect on pain and motility. The results obtained were similar to those obtained with FANS (for example Voltaren® Novartis AG) but at the same time still less effective than nonviable amniotic membranes (Amniex® Mastelli S.r.l., Sanremo, Imperia, IT) (Surace A, Previtera A M, Mineo G, Barberis F. Valutazione dell'efficacia degli estratti placentari sulla sintomatologia algica dell'artrosi. Ortopedia e Traumatologia Oggi 1987; 7: 47-56).

Preliminarily, the same inventors had tested Placentex® (Mastelli S.r.l., Sanremo, Imperia, IT) for the treatment of inflammatory joint diseases, not of the auto-immune rheumatic type, with the purpose of inducing the proliferation and the differentiation of the local stem cells and therefore determining the repair of the joint tissues.

In particular, patients suffering from acute and chronic tendonitis with tenosynovitis by itself or associated with arthrosis of the small and medium joints had been considered. Patients suffering from auto-immune rheumatic diseases and those who had undergone periarticular infiltration of corticosteroids in the previous three months were excluded.

Diseases of auto-immune origin were excluded since they are characterised by different etiology.

For example, excluded auto-immune diseases are rheumatoid arthritis, Sjogren's syndrome, systemic lupus erythematosus, psoriatic arthritis, polymyositis, scleroderma and arthritis associated with systemic auto-immune diseases.

32 patients were selected and treated with intra and peri-articular infiltration of PDRN and mepivacaine 2% as local anaesthetic. Of the 32 patients, 28 were treated with one infiltration per week until there was a significant reduction of symptoms. Of the other four, two left the study because they were non-responsive, one for hospitalization for another disease and one dropped out of the procedure of their own accord.

The patients had shown improvements in terms of pain, motility and joint swelling. The effect was comparable to that of FANS and/or non-crystallized corticosteroids, but the effects of PDRN were slower, although no side effects or adverse reactions of any kind were reported.

No significant structural radiological variations were noted after therapy in patients that had chronic osteoarticular morphological alterations before treatment.

These preliminary results showed that PDRN causes a biological stimulation on the tissues but it has the drawback of being not very long-lasting. PDRN is rapidly absorbed and, indeed, the maximum effect is obtained between 3-5 days from the infiltration to then quickly diminish in the following days.

It is these pharmacodynamic drawbacks that reduces its potential use in regenerative medicine to induce repair of joint tissues.

Therefore, there is a particularly great need to improve the pharmacokinetics of PDRN in order to be able to make effective use of it on degenerative joint diseases (DJD).

It is also well known in the art that surgical trauma determines some modification on biological microenvironment often capable to induce basic reparative transformation. Products of necrosis and O₂ deficiency lead to increase production and activation of local GFs. Specific microenvironmental cues regulate self-renewal and differentiation capabilities. Oxygen is an important component of the cellular microenvironment, serving as both metabolic substrate and signaling molecule. Oxygen has been shown to have a variety of effects on embryonic and adult stem cells. The role of hypoxia in regulating stem cell biology, specifically focusing on growth, maintenance of pluripotency, differentiation, and production of growth factors is becoming more and more relevant (Abdollahi H, Harris L J, Zhang P et al. The Role of Hypoxia in Stem Cell Differentiation and Therapeutics. J. Surg. Res. 2009 Oct. 24).

Trauma necrosis and hypoxia cause local increase of GFs. Some of these GFs as VEGF, FGF 1-2, TGF-beta1 have particular meaning. In fact they can promote an increase of vascular permeability and endothelial cell proliferation, fibroblast chemotaxis, proliferation angiogenesis and matrix deposition.

TGF-beta 1 is involved in granulocyte, macrophage, lymphocyte, fibroblast and smooth muscle cell chemotaxis. It is also implicated in tissue inhibitors of metalloproteinases synthesis (TIMPs) and Matrix metalloproteinases production inhibition.

Most recently TGF-beta1 has been associated with regeneration of articular cartilage. Although transforming growth factor beta1 (TGFbeta1) is known to be a potent inhibitor of proliferation in most cell types, it accelerates proliferation in certain mesenchymal cells, such as articular chondrocytes and nucleus pulposus cells. However, the precise cell cycle progression and molecular mechanisms by which TGF beta1 stimulates cell growth remains unclear (Nakai T, Mochida J, Sakai D. Synergistic role of c-Myc and ERK1/2 in the mitogenic response to TGF beta-1 in cultured rat nucleus pulposus cells. Arthritis Res. Ther. 2008; 10(6):R140).

Furthermore increasing local concentration of basic Fibroblast growth factor (bFGF), Fibroblast growth factor 2 (FGF2), has been associated with increased proliferation of Multipotent mesenchymal stem cells (MSCs) (Rider D A, Dombrowski C, Sawyer A A et al. Autocrine fibroblast growth factor 2 increases the multipotentiality of human adipose-derived mesenchymal stem cells. Stem Cells 2008; 26(6):1598-608).

It has been lately identified a new Hypoxia-inducible factor (HIF) modulator that demonstrated effects over stem cell differentiation status. The biology of the alpha subunits of Hypoxia-inducible factors (HIF alpha) has expanded from their role in angiogenesis to their current position in the self-renewal and differentiation of stem cells (Moreno-Manzano V, Rodríguez-Jiménez F J, Aceña-Bonilla J L et al. FM19G11, a new hypoxia-inducible factor (HIF) modulator, affects stem cell differentiation status. J. Biol. Chem. 2010; 285(2):1333-42).

Heat shock proteins (HSP) are a class of functionally related proteins whose expression is increased when cells are exposed to elevated temperatures or other stress (De Maio A. Heat shock proteins: facts, thoughts, and dreams. Shock 1999; 11 (1): 1-12). This increase in expression is transcriptionally regulated. The dramatic up regulation of the heat shock proteins is a key part of the heat shock response and is induced primarily by heat shock factor (Wu C. Heat shock transcription factors: structure and regulation. Annu. Rev. Cell. Dev. Biol. 1995; 11: 441-69).

The mechanism by which heat-shock (or other environmental stressors) activates the heat shock factor has not been determined. However, some studies suggest that an increase in damaged or abnormal proteins brings HSPs into action. Consequently, the heat shock proteins are also referred to as stress proteins and their up regulation is sometimes described more generally as part of the stress response (Santoro M G. Heat shock factors and the control of the stress response. Biochem. Pharmacol. 2000; 59 (1): 55-63).

Heat-shock proteins are named according to their molecular weight. So Hsp60, Hsp70 and Hsp90 (the most widely-studied HSPs) refer to families of heat shock proteins on the order of 60, 70 and 90 kilodaltons in size, respectively (Li Z, Srivastava P. Heat-shock proteins. Curr. Protoc. Immunol. 2004; Appendix 1: Appendix 1T). The small 8 kilodalton protein ubiquitin, which marks proteins for degradation, also has features of a heat shock protein (Raboy B, Sharon G, Parag H A, Shochat Y, Kulka R G. Effect of stress on protein degradation: role of the ubiquitin system. Acta Biol. Hung. 1991; 42 (1-3): 3-20).

Beginning in the mid-80s, researchers recognized that many HSPs function as molecular chaperones and thus play a critical role in protein folding, intracellular trafficking of proteins, and coping with proteins denatured by heat and other stresses.

The fundamental function of HSPs is expressed as up regulation in stress. Production of high levels of heat shock proteins can be triggered by exposure to different kinds of environmental stress conditions, such as infection, inflammation, exercise, exposure of the cell to toxins (ethanol, arsenic, trace metals and ultraviolet light, among many others), starvation, hypoxia, or water deprivation (Santoro M G. Heat shock factors and the control of the stress response. Biochem. Pharmacol. 2000; 59 (1): 55-63).

Heat shock proteins function as intra-cellular chaperones for other proteins. They play an important role in protein-protein interactions and maintenance such as folding and assisting in the establishment of proper protein conformation and prevention of unwanted protein aggregation. By helping to stabilize partially unfolded proteins, HSPs aid in transporting proteins across membranes within the cell (Walter S, Buchner J. Molecular chaperones-cellular machines for protein folding. Angew. Chem. 2002; 41(7): 1098-113, Borges J C, Ramos C H. Protein folding assisted by chaperones. Protein Pept. Lett. 2005; 12 (3): 257-61).

Heat-shock proteins also occur under non-stressful conditions, simply monitoring the cell's proteins. Some examples of their role as “monitors” are that they carry old proteins to the cell's “recycling bin” and they help newly synthesized proteins fold properly.

These activities are part of a cell's own repair system, called cellular stress response or heat-shock response.

Extracellular and membrane bound heat-shock proteins, especially Hsp70 are involved in binding antigens and presenting them to the immune system (Nishikawa M, Takemoto S, Takakura Y. Heat shock protein derivatives for delivery of antigens to antigen presenting cells. Int. J. Pharm. 2008; 354 (1-2): 23-7).

The functions of HSP, which are typically associated with stress response and tolerance, are well characterized in differentiated cells, while their role in stem cells remains unclear.

Self-renewal and differentiation of stem cells are tightly regulated processes subject to intrinsic and extrinsic signals. Molecular chaperones and co-chaperones, especially heat shock proteins, are ubiquitous molecules involved in the modulation of protein conformational and complexation states. Stem cells exhibit increased stress tolerance and concomitant high levels of chaperone expression (Prinsloo E, Setati M M, Longshaw V M, Blatch G L. Chaperoning stem cells: a role for heat shock proteins in the modulation of stem cell self-renewal and differentiation? Bioessays 2009; 31(4):370-7).

Wang observed that over expression of Hsp20 protected mesenchymal stem cells (MSCs) against cell death triggered by oxidative stress in vitro. The mechanisms contributing to the beneficial effects of Hsp20 were associated with enhanced Akt activation and increased secretion of growth factors (VEGF, FGF-2, and IGF-1) (Wang X, Zhao T, Huang W et al. Hsp20-engineered mesenchymal stem cells are resistant to oxidative stress via enhanced activation of Akt and increased secretion of growth factors. Stem Cells 2009; 27(12):3021-31).

Drawing on these historical experiences and considering the new biological knowledge about Regenerative Medicine, we began treating Degenerative Joint Disease or Osteoarthritis with a new approach based on anatomical joints restoring method, using a gel-repairer effective as stimulator of innate stem cells.

Similarly to other Authors (Surace A, Previtera A M, Mineo G, Barberis F. Valutazione dell'efficacia degliestratti placentari sulla sintomatologia algica dell'artrosi. Ortopedia e Traumatologia Oggi 1987; 7: 47-56), we started by using PDRN (Placentex Integro® 5,625 mg by Mastelli Officina Biofarmaceutica) on DJD during the 1980's but we observed unsatisfactory response in terms of tissue repairing detectable in clinical, radiological and ultrasonography ways. In conclusion Placentex Integro® seemed to cause a short stimulation on tissue before it was absorbed: the effect peaks within 3-5 days of the injection and decreases rapidly in the following days. This pharmaco-dynamic property reduces the potential use of the drug for tissue repair, which often needs a longer biological stimulation to activate the innate reparative mechanisms.

Moreover clinical results had shown that PDRN was less effective than Amnios (as Amniex®) membranes.

We considered three possible main causes for this evidence:

1) fast diffusion, low PDRN absorption and weak stimulation over local innate stem cells, 2) lack of surgical trauma, 3) different nature of substances used in the treatment.

Then we compared the results of the procedures of PDRN alone to PDRN and blood, the later resulted in: 1) improvement in terms of pain and articular motion, evaluated by 68% of the patients as good outcome of the therapy, and stable after 12 months (vs. 43% of PDRN alone), 2) the follow-up showed better long term results, 3) the average number of infiltration was reduced by 40%, 4) there were fewer therapeutic failures.

The closest state of the art had mainly investigated in vitro and on animal models the ability of PDRN to stimulate cell proliferation (Guizzardi et al., 2003; Guizzardi et al., 2007, supra).

The purpose of the research forming the basis of the present invention was to make the basic biochemical elements present in the haematic component in a highly concentrated form.

Moreover, from the point of view of industrial scale application it is advantageous to have a stable haematic component that is not necessarily associated with phenomena of heterological rejection.

In order to obtain an end product in which the polynucleotides and the Heat Shock Proteins are present in high concentrations, the starting haematic component must contain at least one blood cell component.

The treatment to which the starting haematic component is subjected allows an end product rich in Heat Shock Proteins and Polynucleotides to be obtained.

The activation of the molecules in the end product takes place directly in the periarticular extracellular microenvironment thanks to local hypoxia, to enzyme mediators and to growth factors present in loco.

Therefore, it must be specified that the haematic component, which has certain starting characteristics, thanks to the treatment to which it is subjected, transforms into an end product rich in fragmented nucleic acids and denatured proteins (Heat Shock Proteins) together with other secondary products of the transformation procedure. These biologically active elements as mediators and cell stimulators are not present in the initial material but they are only in the end product.

The heat shock process carried out from low (−16/20 C.°) to high temperatures (+120-160 C.°) to which the haematic component is subjected, makes it possible to state that the end product is rich in denatured Heat Shock Proteins.

Although the nature and amount of HSP present and actually effective in inducing proliferation and differentiation stimulation on the cells has not yet been directly determined, based on the literature on this subject, it is realistically possible to state that in the end product there is a substantial amount of HSP with a molecular weight of between 8 and 110 kilodalton. Indeed, in the literature the fractions of HSP of between 8 and 20 kilodalton are amongst those most quoted as molecules capable of activating and regulating the stem compartment (De Maio A. Heat shock proteins: facts, thoughts, and dreams. Shock 1999; 11 (1): 1-12; Wu C. Heat shock transcription factors: structure and regulation. Annu. Rev. Cell. Dev. Biol. 1995; 11: 441-69; Santoro M G. Heat shock factors and the control of the stress response. Biochem. Pharmacol. 2000; 59 (1): 55-63; Li Z, Srivastava P. Heat-shock proteins. Curr. Protoc. Immunol. 2004; Appendix 1: Appendix 1T; Raboy B, Sharon G, Parag H A, Shochat Y, Kulka R G. Effect of stress on protein degradation: role of the ubiquitin system. Acta Biol. Hung. 1991; 42 (1-3): 3-20; Walter S, Buchner J. Molecular chaperones-cellular machines for protein folding. Angew. Chem. 2002; 41(7): 1098-113; Borges J C, Ramos C H. Protein folding assisted by chaperones. Protein Pept. Lett. 2005; 12 (3): 257-61; Nishikawa M, Takemoto S, Takakura Y. Heat shock protein derivatives for delivery of antigens to antigen presenting cells. Int. J. Pharm. 2008; 354 (1-2): 23-7; Prinsloo, Setati M M, Longshaw V M, Blatch G L. Chaperoning stem cells: a role for heat shock proteins in the modulation of stem cell self-renewal and differentiation? Bioessays 2009; 31(4):370-7; Wang X, Zhao T, Huang W et al. Hsp20-engineered mesenchymal stem cells are resistant to oxidative stress via enhanced activation of Akt and increased secretion of growth factors. Stem Cells 2009; 27(12):3021-31).

### OBJECT OF THE INVENTION

The object of the present invention is a haematic component obtainable by the process comprising the following steps a) subjecting blood or a blood derivative, wherein said blood derivative comprising at least one cellular component, to thermal treatment at a temperature between −10 e-50° C. for at least 12-16 hours and b) subjecting the product obtained in step a) to thermal treatment at a temperature between 100 and 200° C. for a time between 60 and 200 minutes.

In one embodiment of the present invention blood is autologous blood.

Said above haematic component is solid.

Another object of the present invention is a composition comprising said haematic component and a fluidifying agent, optionally comprising PDRN and/or at least one Heat Shock Protein and further optionally comprising mesenchimal stem cells.

Suitable fluidifying agents are well known to the person with ordinary skills in the field of pharmacology.

The compositions comprising a fluidifying agent are in a fluid form.

A further object of the present invention is a composition comprising said haematic component and a thickener, optionally comprising PDRN and/or at least one Heat Shock Protein.

Said compositions are in the form of a gel.

The gel is prepared by following the process comprising the following steps:
a) diluting PDRN or PDRN and at least one heat shock protein with the haematic component as disclosed in claim 1;
b) adding a thickener to the composition obtained in a) and subsequently mixing;
c) putting the composition obtained in b) in plates and subsequently treating for at least 12-16 hours at a temperature between −10 and −50° C.;
d) sterilizing at a temperature between 100 and 200° C. for a time between 60 and 200 minutes.

A further object of the present invention is the use of the said haematic component as a medicament.

A further object of the present invention is the use of the above haematic component and of the above compositions for the preparation of a medicament for the treatment of not-autoimmune degenerative joint disease.

A further object of the present invention is a kit comprising anyone of the above compositions and means for intra-articular and peri-articular administration.

It is also an object of the present invention a method for the treatment of not-autoimmune degenerative joint disease comprising the intra-articular and peri-articular administration of the haematic component or anyone of the above compositions.

Further details of the present invention are obvious in light of the detailed description and the following examples and from the drawings wherein:

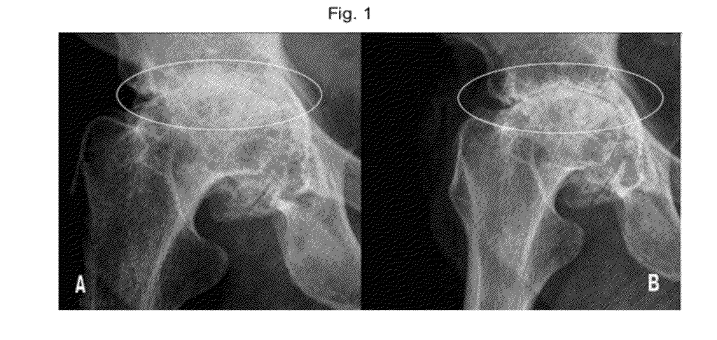

FIG. 1 shows a right hip; FIG. 1A shows IV grade K&L before treatment; FIG. 1B shows? grade K&L after treatment (6^{th} month follow-up).

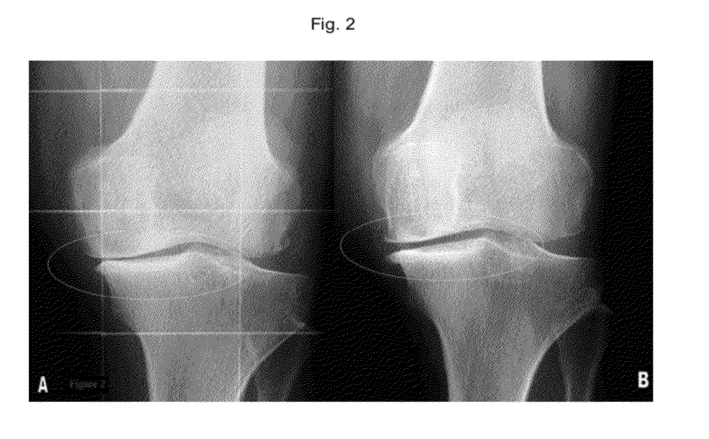

FIG. 2 shows a left knee, FIG. 2A shows a III grade K&L before treatment; FIG. 2B II grade K&L after treatment (20^{th} month follow-up).

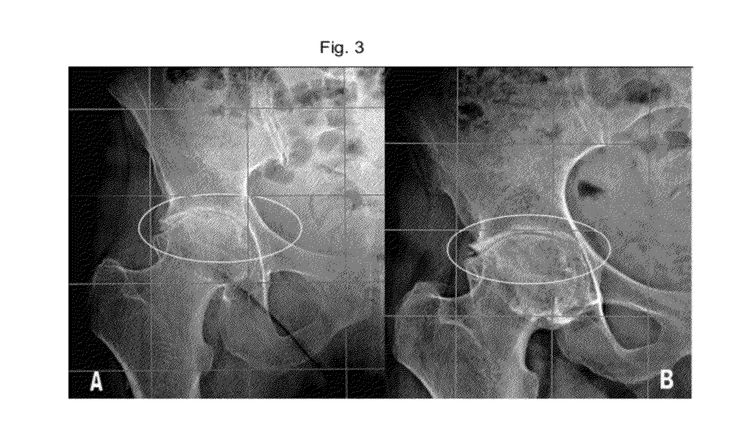

FIG. 3 shows a right hip, FIG. 3A IV grade K&SL before treatment; FIG. 3B III grade K&L after treatment (43^{th} month follow-up).

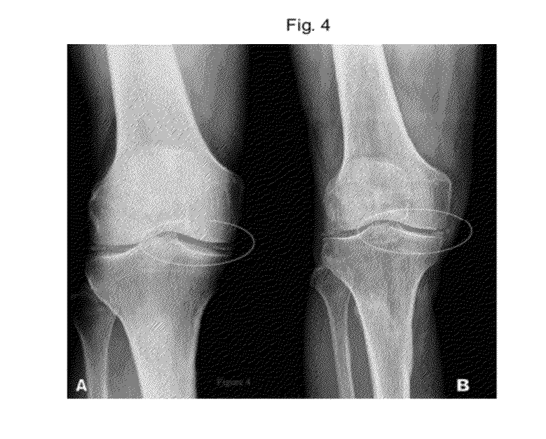

FIG. 4 shows a right knee with severe chondrocalcinosis in DJD, FIG. 4A shows before treatment; FIG. 4B shows after treatment (6^{th} month follow-up); mainly the medial compartment of the knee had been treated.

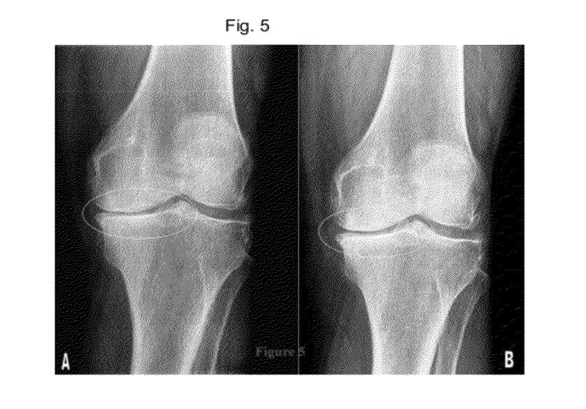

FIG. 5 shows a left knee; FIG. 5A shows III grade K&L before treatment; FIG. 5B shows II grade K&L after treatment (7^{th} month follow-up).

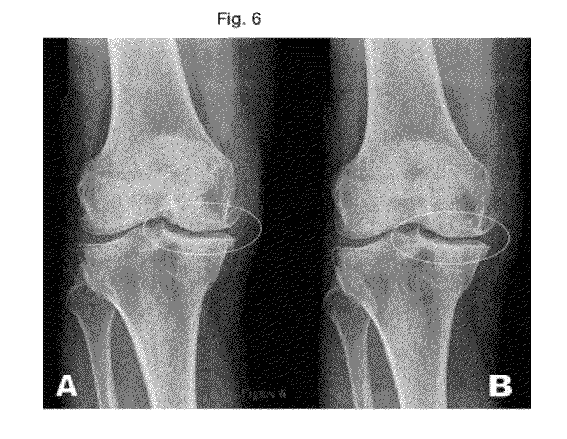

FIG. 6 shows a right knee, FIG. 6A shows III grade K&L before treatment; FIG. 6B shows III grade K&L after treatment (8^{th} months follow-up); clinical improvement was more evident than radiological.

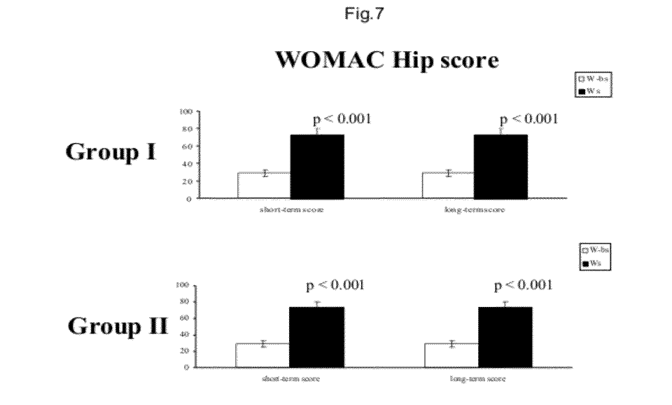

FIG. 7 shows a WOMAC hip score before and after treatment at short and long term, the data were expressed as mean±SD. Differences were considered significant at the level of p<0.01; □W-bs=WOMAC basic score; ▪Ws=WOMAC score (short/long term).

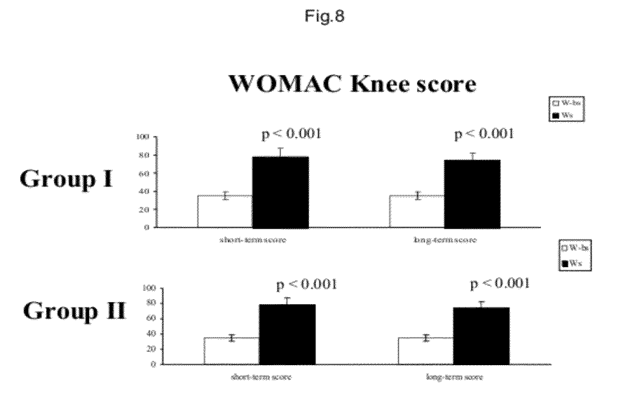

FIG. 8 shows a WOMAC knee score before and after treatment at short and long term, the data were expressed as mean±SD. Differences were considered significant at the level of p<0.01; □W-bs=WOMAC basic score; ▪Ws=WOMAC score (short/long term).

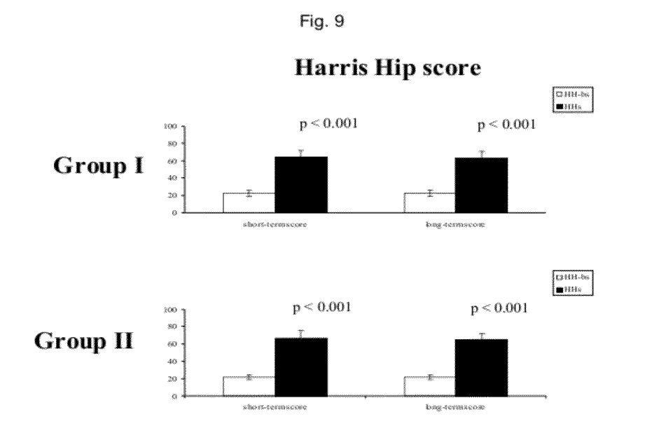

FIG. 9 shows a Harris Hip score before and after treatment at short and long term. The data were expressed as mean±SD; differences were considered significant at the level of p<0.01; □HH-bs=Harris Hip basic score, ▪HHs=Harris Hip score (short/long term).

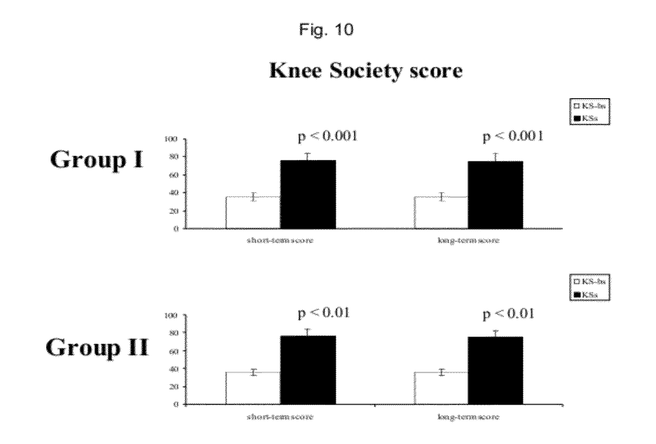

FIG. 10 shows a Knee Society score before and after treatment at short and long term; the data were expressed as mean±SD. Differences were considered significant at the level of p<0.01; □KS-bs=Knee Society basic score; ▪KSs=Knee Society score (short/long term).

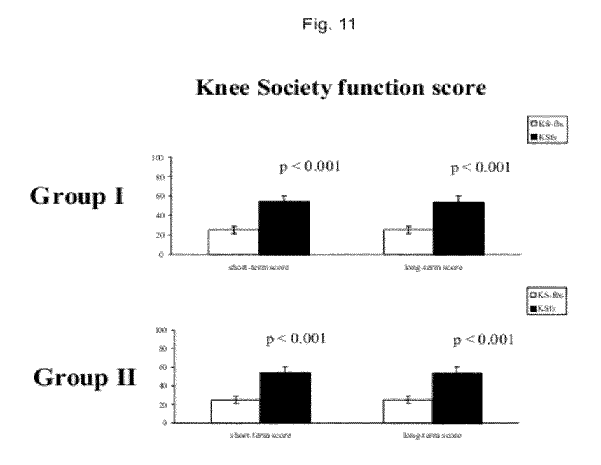

FIG. 11 shows a Knee Society function score before and after treatment at short and long term; the data were expressed as mean±SD. Differences were considered significant at the level of p<0.01; □KS-fbs=Knee Society function basic score; ▪KS fs=Knee Society function score (short/long term).

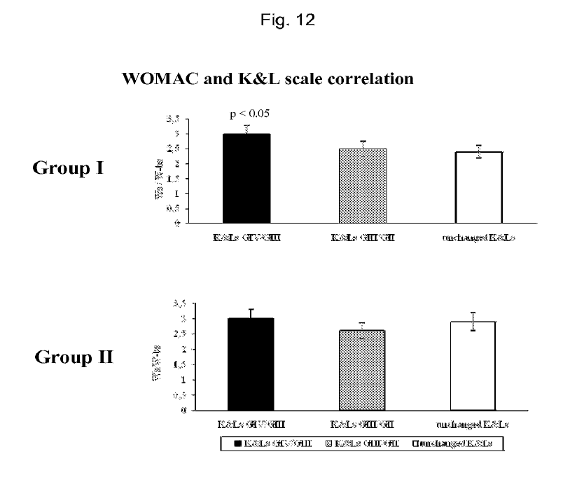

FIG. 12 shows a correlation WOMAC classification versus Kellgren and Lawrence scale. Columns represent the average ratio Ws/W-bs in respect to both downstaging and maintenance of K&L scale; data were expressed as mean±SD. Differences were considered significant at the level of p<0.05.

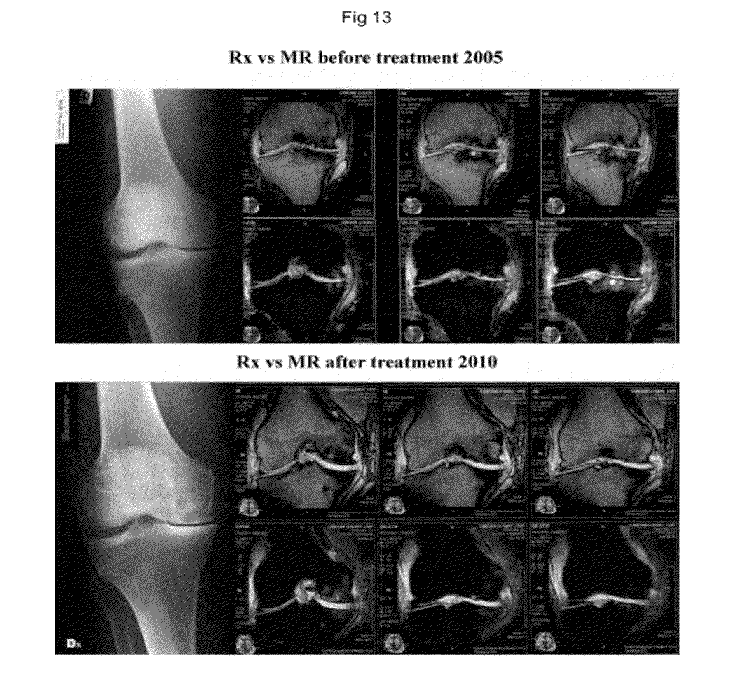

FIG. 13 shows the comparison of a Rx and a MR of the right knee of a 53 years old patient before and after treatment.

### DETAILED DESCRIPTION OF THE INVENTION

The following definitions are disclosed:

ASA means American Society of Anesthesiologists Classification of Preoperative Risk.
DJD means Degenerative Joint Disease.
HH-bs means Harris Hip basic score.
HHs means Harris Hip score; in short or long term.
KS-bs means Knee Society basic score.
KSs means Knee Society score, in short or long term.
KS-fbs means Knee Society function basic score.
KSfs means Knee Society function score, in short or long term.
K&L means Kellgren and Lawrence Scale.
MR means Magnetic Resonance.
NSAIDs means Nonsteroidal anti-inflammatory drugs.
OA means osteoarthritis.
PDRN means Polydeoxyribonucleotides.
Patients means patients.
WOMAC means Western Ontario and McMaster Universities.
W-bs means WOMAC basic score.
Ws means WOMAC score, in (short or long term.
By PDRN we mean polydeoxyribonucleotide, a linear polyanion, the monomer units of which consist of deoxyribonucleotides bonded by a phosphodiester bond.

In an embodiment, PDRN has an average molecular weight of about 350 KD.

In another embodiment PDRN consists of a mixture of thermostable deoxyribonucleotides consisting of polymers of variable length of between 50 and 2000 bases and of nucleosides.

Said mixture can, for example, be obtained from purification of salmon and trout sperm.

By haematic component we mean blood, or a derivative thereof comprising at least one blood cell component (depicted element) obtained through the process the foresees in stage a) heat treatment at a temperature of between −10 and −50° C., preferably between −10 and −20° C., for at least 12-16 hours, preferably 12 hours, and in stage b) heat treatment at a temperature of between 100 and 200° C., preferably between 140 and 160° C., for a time of between 60 and 200 minutes, preferably between 60 and 120 minutes.

The haematic component can be selected among the group consisting of whole blood, concentrate of red blood cells, platelet jelly.

Preferably, the haematic component is whole blood.

The whole blood can belong to any blood type or group since the process for its preparation results in any antibody reaction being cancelled out.

Said haematic component can be in fluid form, preferably liquid, able to be obtained for example by adding a suitable fluidifying agent, like a proteolytic solution with trypsin, or through another appropriate medium known to the man skilled in the art.

By degenerative joint disease (DJD) we mean a chronic degenerative disease of the joint cartilage that extends involving all of the tissues that constitute the joint. It is a degenerative-inflammatory articular and periarticular disease, excluding auto-immune rheumatic diseases.

Examples of degenerative joint disease (DJD) are constitutional arthrosis, postural arthrosis, post-traumatic arthrosis or malformation arthrosis, characterised by chronic capsule-ligament inflammation and initial joint damage (capsule-ligament thickening and cartilage erosion) or advanced joint damage (contact between the joint surfaces with serious cartilage erosion and subchondral bone oedema) or ankylosis, tendinitis, tendinosis, chronic tenosynovitis, localised, by itself or combined with arthrosis.

Degenerative joint diseases of auto-immune origin are excluded from the present invention.

By thickener we mean a pharmaceutically acceptable additive having gelling action that is inert at the temperatures of preparation of the gel object of the present invention.

The thickener is selected among the group consisting of glycerol, fish glue, polyethylene glycol, agar-agar, cellulose, carob flour, sodium alginate, carrageenan, pectin, tragacanth, gum arabic, tara gum, konjac gum, xanthan gum, gellan gum, microcrystalline cellulose, methylcellulose, phosphates, polyphosphates, amides, proteoglycans and hyaluronic acid.

In a preferred embodiment the thickener is glycerol since it is stable at the sterilization temperatures.

By mesenchymal stem cell we mean a pluripotent cell, i.e. one that transforms only into some types of tissue according to the germ line that it has acquired. The mesenchymal stem (MSC) differs from bone, cartilage, muscle and adipose tissues.

Adipose tissue and the bone marrow are recognised as reservoirs of mesenchymal stem cells (MSC).

Heat shock protein (HSP) means a protein belonging to the class of functionally related proteins whose expression is increased when cells are exposed to elevated temperatures or other stress (De Maio A. Heat shock proteins: facts, thoughts, and dreams. Shock 1999; 11 (1): 1-12).

The dosage of each active ingredient is evaluated by the man skilled in the art so that it is therapeutically effective and at the same time does not exhibit toxic effects or side effects like pain from expansion and from ischemic suffering of the tissue, disseminated coagulation, fever.

In compositions comprising PDRN, haematic component and thickener it is preferred for the ratio between PDRN and haematic component to be 1:1 since the gel that is obtained has the best characteristics for use, said amount being roughly two and a half times the amount of thickener.

The haematic component, in fluid form, preferably liquid, by itself or in combination with PDRN and/or at least one heat shock protein and also with mesenchymal stem cells is therapeutically effective since the haematoma generated and the detachment of the tissues traps the macromolecules of PDRN and delays their release, increasing the exposure times of the active ingredient on the damaged tissues.

Moreover, the local haemolysis results in the release of different substances that can enhance the biostimulant effectiveness of PDRN like the increase in nucleic acids in the extracellular matrix, the activation of growth-factors, cytokine and endorphin.

The gel allows a prolonged stimulation action on the local stem cells, promoting the repair mechanism. Moreover, the gel acts as a scaffold to guide the stem cells towards adhesion, proliferation and differentiation.

The gel stimulates the tissues to repair causing the proliferation of fibroblasts and the production of elastin and collagen in a correct ratio, having the clinical result of an appreciable increase in flexibility of the articular capsule and of the ligaments with an increase in overall compliance, reduction in intra-articular pressure and in pain.

Moreover, the gel stimulates the periosteal reaction with proliferation of the osteoblasts until bone repair is induced.

Finally, the gel causes the increase in matrix proteins of the cartilage compartment perhaps induced by the activation of chondroblasts.

The results show that the repair gel offers a new therapeutic option for the treatment of arthrosis at any stage of the disease, from chronic inflammation with joint damage to ankylosis.

It is also true that the treatment with the gel object of the present invention does not rule out other subsequent treatment options.

The man skilled in the art knows very well that stem cells are cells that upon each division create one cell identical to itself and one commissioned cell, i.e. oriented towards a differentiation line. With this asymmetric division the number of stem cells (stem reservoir) is kept unchanged, whereas the commissioned cells by dividing and differentiating create the mature cells that constitute the tissues. The stem cells are distinguished into totipotent, i.e. capable to transforming into any type of tissue and into pluripotent that transform just into some types of tissue according to the germ line that they have acquired. The germ lines are, for example, mesenchymal, epithelial, neuroectodermal. In particular from the mesenchymal line the tissues are differentiated: bone, cartilage, tendons, muscles and sheaths and adipose tissue. Unipotent cells, on the other hand, can only create one cell line.

Adipose tissue and bone marrow are recognised as reservoirs of mesenchymal stem cells (MSC).

It is known in literature that adipose tissue contains pluripotent stem cells that if suitably activated have the ability to follow the differentiation paths foreseen in the mesenchymal germ line and therefore these cells can develop fibroblasts, osteoblasts and chondroblasts (Raposio E, Guida C, Coradeghini R et al. In vitro polydeoxyribonucleotide effects on human pre-adipocytes. Cell Prolif. 2008; 41(5): 739-54).

In order to carry out their effect, adipose stem cells must find a suitable environment for their survival capable or orienting their proliferation and differentiation development, which they find in the binary mixture between PDRN and haematic component.

In a further embodiment of the invention the binary composition of PDRN and haematic component in fluid, preferably liquid, form as described previously, is added to with mesenchymal stem cells (MSC).

Preferably the mesenchymal cells (MSC) come from adipose tissue or from bone marrow and even more preferably they are adult adipose stem cells.

Preferably, the adult adipose stem cells are added in the form of subcutaneous adipose tissue.

In an embodiment, the PDRN and the haematic component are in equal quantities whereas the quantity of mesenchymal stem cells (MSC) is half that of the other components.

It has been observed that in the preparation of the mixture object of the present invention the step of in vitro cultivation is not necessary, allowing the simple and direct use of the mesenchymal stem cells (MSC).

Indeed, it has been noted that the proliferation and the cell differentiation during in vitro cultivation reduce the incubation and survival ability of mesenchymal stem cells (MSC).

This composition is prepared by adding PDRN, preferably in equal quantity to the haematic component as described previously and adding the adult adipose stem cells, preferably in a quantity equal to half the PDRN and haematic component.

Any or all of the previous embodiments can be provided as kits.

In an embodiment a kit is formed from the haematic component by itself or added to with the thickener or with PDRN or the composition of haematic component, PDRN and thickener together with suitable mediums for intra-articular and/or peri-articular administration.

Each kit includes the appropriate package and the relative instructions.

In an embodiment of the invention the method for therapeutically treating degenerative joint disease (DJD) is carried out mini-invasively and in an ambulatory manner.

The method foresees the following steps:

a) identifying the joint area to be treated;
b) local anaesthetic;
c) incision;
d) blunt dissection;
e) peri-articular application of the gel object of the invention
f) suture;
in which the procedure is repeated weekly until clinical stabilization.

In a further embodiment, the method for therapeutically treating degenerative joint disease (DJD) foresees the following stages:

a) the donor area is disinfected and the local anaesthetic is infiltrated;
b) the liposuction cannula needle is introduced mounted on a heparinised syringe and the subcutaneous fat is sucked up;
c) with the same syringe the liposuction cannula needle is removed and it is replaced with a blood drawing needle and venous blood is drawn and PDRN is added to it;
d) the receiving joint area is disinfected and anaesthetized;
e) the needle of the syringe in b) is changed and the compound preparation of adipose tissue, blood and PDRN is infiltrated periarticularly.

In a further embodiment, the method for therapeutically treating degenerative joint disease (DJD) foresees the following stages:

a) the donor area is disinfected and the local anaesthetic is infiltrated;
b) the bone marrow biopsy needle is introduced and bone marrow is drawn;
c) the bone marrow biopsy needle is removed and it is replaced with a blood drawing needle and venous blood is drawn and PDRN is added to it;
d) the receiving joint area is disinfected and anaesthetized;
e) the composition of bone marrow, blood and PDRN is infiltrated periarticularly.

The following example illustrate the invention further.

### Example 1

The patients were included in the study through a questionnaire (score from 0 to a maximum of 14) to qualitatively and quantitatively define the pain and functional impotence of the joint together with the clinical evaluation (score 0-6) with X-rays, magnetic resonance and rarely echo-tomography. The same questionnaire and the same clinical examinations were also used to evaluate the short and long term outcome of the treatment.

Thus the patients were selected based on four main parameters:

Diagnosis: tendinitis, tendinosis and chronic tenosynovitis, localised, by themselves or combined with arthrosis and exclusion of patients carrying systemic and auto-immune diseases.

Stage of advancement of the disease: chronic tendinitis with or without slight signs of structural damage; arthrosis level I-II (chronic inflammation with initial joint structural damage).

Location of the joint: joints of small and medium size (hand, foot, wrist, angle, elbow).

Patients undergoing therapies based on crystallized corticosteroids carried out within the last 3 months were excluded.

All of the patients were treated with the composition comprising 3 cc of PDRN (Placentex® integro 5.625 mg Mastelli S.r.l.) and 4 cc of autologous whole blood, subjected first to heat treatment at a temperature of between −10 and −20° C., for 12 hours, and then to heat treatment at a temperature of between 140 and 160° C., for a time of between 60 and 120 minutes.

The peri-intra-articular infiltration was preceded by the infiltration of mepivacaine 2% as local anaesthetic.

The infiltration of the preparation was repeated every fifteen days until completion of the clinical profile.

The evaluation of the outcome of the treatment was set at 3 (short term) and 12 months (long term) from the end of the therapy.

181 patients were selected and treated, divided into two sub-groups per diagnosis: 102 patients with tendinitis and chronic tenosynovitis without arthrosis (group A) and 79 patients with tendinitis and chronic tenosynovitis with arthritis (group B).

The results obtained were compared with those with PDRN alone.

It was found in favour of the mixture, according to the present invention, consisting of PDRN (Placentex® integro 5,625 mg Mastelli S.r.l.) and whole blood, that there was an improvement in terms of pain and joint motility evaluated as good by 72% of the patients treated (compared to 50% of the patients treated with PDRN alone) with a more significant improvement in patients of group B. After 12 months the result was better and more stable, the number of therapeutic infiltrations was less than 40%, the therapeutic failure rate was less, rehabilitation therapy improved the results over the long term to a greater extent than PDRN alone.

### Example 2

A repair gel was prepared consisting of 16 cc of blood diluted in 16 cc of Placentex® integro 28.125 mg (Mastelli S.r.l., Sanremo, Imperia, IT) and 6 cc of liquid glycerol for a total of 38 cc of preparation.

In the preparation process the aforementioned mixture was briefly mixed and placed in two sterile Petri dishes (19 cc of preparation per dish) to be arranged in a freezer for at least 12 h at a temperature of −16/20° C. After freezing the dishes were introduced into a dry sterilizer at a temperature of 140/160° C. for 120 minutes. A compound with a gellified appearance, that is dense, dark brown in colour, able to be separated into fragments with a scalpel blade, odourless, non-deteriorating, able to be conserved in the production Petri dish is thus obtained by adding 2 cc of povidone-iodine.

The patients were included in the study through a questionnaire (score from 0 to a maximum of 14) to qualitatively and quantitatively define the pain and functional impotence of the joint together with the clinical evaluation (score 0-6), and the radiological examinations (X-rays, magnetic resonance, echo-tomography). The same questionnaire and the same clinical examinations were also used to evaluate the short and long term outcome of the treatment.

Patients with constitutional arthrosis, postural arthrosis, post-traumatic arthrosis or malformation arthrosis, characterised by chronic capsule-ligament inflammation and initial joint damage (capsule-ligament thickening and cartilage erosion) or advanced joint damage (contact between the joint surfaces with serious cartilage erosion and subchondral bone oedema) or ankylosis were included.

Patients who had taken corticosteroid therapy in the past 3 months, or were suffering from auto-immune generalised acute arthritis or osteomyelitis or had a prothrombin time (INR) greater than 3 were excluded.

The gel was glued peri-articularly after local anaesthesia with mepivacaine 2%+naropin, incision of about 5 mm and blunt dissection, followed by suture.

The procedure was repeated weekly until clinical assessment with a preliminary evaluation of the clinical results after the first 3 therapy sessions.

948 patients were treated for a total of 6878 procedures.

Of these, 792 patients entered into variable duration evaluation of from 6 months to 5 years.

The percentage of patients that responded to the therapeutic treatment was 85% (673 patients) with an average age of 62 years.

After 6 months of treatment, based on the questionnaire and the clinical examination (with the respective scores), of the 673 patients that gave a clinical response to the therapy, 80% rated the result as “good”, 15% rated the result as “excellent” and 5% rated the result as “sufficient”.

Of the remaining 119 patients (15%), 7% considered the procedure to have failed whereas 8% were unable to judge the result of the therapy.

The complications that occurred were, 1.8% of the time infections of the surgical wound and 5% of the time flaws like hyperpigmentation or dystrophic wound.

There is a coherent correlation between the clinical improvement and the radiological images.

The clinical result after three months from the end of the therapy was considered stable.

After 1 year from the end of the procedure, 76% of patients rated the end result as good and this allowed them to return to a satisfactory quality of life.

10% of these patients had requested some additional therapeutic applications during the first year of follow-up, with an average of three additional therapeutic sessions. 90% of these patients then reported that they had stabilised with an end result rated as good.

Only 40% of patients underwent rehabilitation therapy after the treatment and in terms of performance and of effectiveness of the therapy over time this group achieved often excellent results.

In the group of patients (5%) that rated the result as “sufficient” in the short term, 70% of these reported a progressive and spontaneous improvement during the first year.

After 3 years 242 patients (36%), and after 5 years 67 patients (10%), considered the result stable in 70% and in 67% of those interviewed, respectively.

It should be noted that some of the patients treated, with an advanced stage of the disease, had already been advised to get a prosthesis, whilst other patients, either due to very poor general conditions or due to diseases that required difficult surgical procedures, had no other therapeutic options. Of these, 7 patients out of 10, who were on the prosthesis waiting list, postponed or rejected prosthetic surgery after the treatment with the gel object of the present invention.

### Example 3

36 patients selected according to the parameters shown in example 1 were treated with autologous adult adipose stem cells added to with the PDRN/blood mixture.

About 1-1.5 cc of sub-cutaneous fat, 3 cc of venous blood treated like in example 1; 5.625 mg of PDRN (Placentex integro).

No laboratory cultivation steps were necessary between the time of drawing and reinoculation of the stem cells.

The evaluation 6 months after the therapy of the first 15 patients treated was carried out using a questionnaire (score from 0 to a maximum of 14) to qualitatively and quantitatively define the pain and functional impotence of the joint together with the clinical evaluation (score 0-6). The same questionnaire and the same clinical examinations were also used to evaluate the short and long term outcome of the treatment.

Of these, 7 patients responded to the treatment rating it good in terms of resolution of the pain and recovery of motility, 5 rated it as excellent, two just sufficient and only one rated the result as bad.

### Example 4

In a sub-group of 5 patients the therapy with the composition of example 1 was also associated sequentially with the therapy of example 2, obtaining surprisingly good results.

### Example 5

The method for therapeutically treating degenerative joint disease (DJD) was carried out mini-invasively and in an ambulatory manner using the following equipment: sterile field, disinfectant, local anaesthetic, scalpel blade n^{o}11, 1 straight Kelly forceps, 1 bayonet forceps, 1 needle holder, scissors, 1 monofilament suture and a sterile medication.

The method foresaw locating the joint area to be treated, followed by local anaesthesia with mepivacaine 2%+naropin. An incision of about 5 mm was then made and blunt dissection was carried out. The gel was placed in a peri-articular manner and the part was sutured. The procedure was repeated weekly until clinical assessment.

### Example 6

The method for therapeutically treating degenerative joint disease (DJD) was carried out using disinfectant (for example povidone-iodine), sterile field, local anaesthesia, for example mepivacaine 2%+naropin, scalpel blade n^{o}11, 20 cc syringe, liposuction micro-cannula-needle, blood drawing needle 22 G, inoculation needle 18 G.

The method foresaw infecting the donor area (hip or iliac fossa), infiltrating about 3 cc of local anaesthetic and introducing the liposuction cannula-needle mounted on a heparinised syringe to suck up about 1-1.5 cc of subcutaneous fat. With the same syringe the liposuction cannula needle was removed and it was replaced with a blood drawing needle to draw 3 cc of venous blood. This was added to with 5.625 mg of PDRN (Placentex® integro 5,625 mg Mastelli S.r.l.);

The receiving joint area was disinfected and anaesthetized. The needle of the syringe in b) was changed with one of 18 G and the compound preparation of 1-1.5 cc of adipose tissue, 3 cc of homologous blood and 3 cc of PDRN (Placentex® integro 5.625 mg Mastelli S.r.l.) was infiltrated periarticularly.

### Example 7

From January 2003 until June 2009 were treated 948 patients for DJD covering virtually all medium and large articulations. The focus of this analysis is on two groups of patients. The first group (I) considered for this study was composed of 86 over eighties patients affected by DJD of the hip or/and knee. The second group (II) was composed of 90 patients around fifty years old affected by the same disease but the causes of DJD were quite different as post-traumatic, congenital hip dysplasia, arthritis induced by severe postural defects.

All patients were referred by orthopedic surgeons and rheumatologists with a clinical diagnosis of DJD or OA in very advanced staging and symptoms with greater than 6 months duration.

The first group of patients was judged high surgical risk for surgical prosthesis (the most were ASA III-IV) and both groups had been non-responders to currently adopted conservative therapies and all of them had become non-responders or intolerants at NSAID's and Corticosteroids.

All patients gave informed consent before entering the study.

Patients who underwent corticosteroids therapy over the last month, INR over 3.5 and affected by acute rheumatic diseases were excluded.

All patients of both groups underwent local treatment with gel-repairer. Gel-repairer is prepared with distressed (low and high temperature) processed Blood, PDRN and a thickening substance.

The quantity of gel that was used to treat the joints depended on the volume of articulation and the thickness of subcutaneous fat. The average quantity of gel used in each point of introduction was 95 mg (range 55-110) for the hip and 42 mg for the knee (range 35-60).

Generally the treatment was performed simultaneously on 2 or 3 areas of the joint previously evaluated by clinical and radiological assessment.

The area needing treatment was injected with local anesthesia composed by both mepivacaine 2% (2 ml) and naropine 10% (2 ml), 4-5 ml in total. A minimal incision (5 mm) was made in order to introduce a Kelly forceps and reach subcutaneous tissue in peri-articular space where the gel was placed, then the wound was sutured.

The treatment was repeated at weekly intervals. Preliminary results were assessed after three procedures.

Non-responders patients were those who did not improve after three treatments.

A subjective patient completed Questionnaire (WOMAC) of Hip and/or Knee was adopted with and two Clinician schedules, filled by the physician, respectively known as Harris Hip score and Knee Society score.

The Western Ontario and McMaster Universities (WOMAC) Osteoarthritis Index is a disease specific, self-administered, health status measure. It probes clinically important symptoms in the areas of pain, stiffness and physical function in patients with osteoarthritis (OA) of the hip and/or knee. The index consists of 24 questions (5 pain, 2 stiffness and 17 physical function). The WOMAC is a valid, reliable and sensitive instrument for the detection of clinically important changes in health status following a variety of interventions (pharmacologic, surgical, physiotherapy, etc.) (Bellamy N. WOMAC: a 20-year experiential review of a patient-centered self reported health status questionnaire. J Rheumatol 2002; 29:2473-6; Soderman P and Malchau H. Validity and reliability of Swedish WOMAC osteoarthritis index: a self-administered disease-specific questionnaire (WOMAC) versus generic instruments (SF-36 and NHP). Acta Orthop Scand 2000; 71:39-46; Jinks C, Jordan K, Croft P. Measuring the population impact of knee pain and disability with the Western Ontario and McMaster Universities Osteoarthritis Index (WOMAC). Pain 2002; 100:55-64; Angst F, Aeschlimann A, Steiner W, Stucki G. Responsiveness of the WOMAC osteoarthritis index as compared with the SF-36 in patients with osteoarthritis of the legs undergoing a comprehensive rehabilitation intervention. Ann Rheum Dis 2001; 60:834-40; Theiler R, Sangha O, Schaeren S, et al. Superior responsiveness of the pain and function sections of the Western Ontario and McMaster Universities Osteoarthritis Index (WOMAC) as compared to the Lequesne-Algofunctional Index in patients with osteoarthritis of the lower extremities. Osteoarthr Cartil 1999; 7:515-9).

Questionnaire was filled by patients at baseline prior to treatment and then during the follow up.

Individual question responses are assigned a score between 0 (extreme) and 4 (None). Individual question scores are then summed to form a raw score ranging from 0 (worst) to 96 (best). Finally, raw scores are normalized by multiplying each score by 100/96. This produces a reported WOMAC Score of between 0 (worst) to 100 (best).

The Harris Hip Score (HHs) is created to evaluate patients' status after hip prosthesis surgery. Questions are grouped into 5 categories: pain, motility, functional activities, physical examination. Score ranges between 0 (worst) to 100 (best) (Hoeksma H L, Van den Ende C H M, Ronday H K, Heering A, Breedveld F C, Dekker J. Comparison of the responsiveness of the Harris Hip Score with generic measures for hip function in osteoarthritis of the hip. Ann Rheum Dis 2003; 62: 935-38).

The Knee Society score (KSs) is subdivided into a knee score that rates only the knee joint itself and a functional score (KSfs) to assess the patient's ability to walk and climb stairs. The knee rating system considers the following main joint parameters: pain, stability and range of motion, flexion contracture, extension lag and misalignment. Thus 100 points will be obtained by a well-aligned knee with no pain, 125 degrees of motion and negligible anteroposterior and mediolateral instability. Patients joint function considers only walking distance, stairs climbing and walking aids. The maximum function score, which is also 100, is obtained by a patient who can walk an unlimited distance and go up and down stairs normally. The form itself is largely self-explanatory: 50 points are allotted for pain, 25 for stability, and 25 for range of motion. Walking ability is expressed in blocks (approximately 100 meters). Stair climbing is considered normal if patient can ascend and descend stairs without holding a railing itself is largely self-explanatory: 50 points are allotted for pain, 25 for stability, and 25 for range of motion (Moonot P, Medalla G A, Matthews D, Kalairajah Y, Field R E. Correlation between the Oxford Knee and American Knee Society Scores at Mid-Term follow-up. J Knee Surg 2009; 22: 226-30).

Patients were classified following the Kellgren and Lawrence Scale (K&L) for radiological assessment of DJD.

The scale defines four pathological degrees for OA: Grade I: doubtful narrowing of joint space and possible osteophytic lipping. Grade II: definite osteophytes, definite narrowing of joint space. Grade III: moderate multiple osteophytes, definite narrowing of joints space, some sclerosis and possible deformity of bone contour (pre-ankylosis).

Grade IV: large ostheophytes, marked narrowing of joint space, severe sclerosis and deformity of bone contour (ankylosis) (Kellgren J H and Lawrence J S. Radiological assessment of osteo-arthrosis, Ann Rheum Dis 1957; 16:494-502; Shiphof D, Boers M, Bierma-Zeinstra S M. Differences in descriptions of Kellgren and Lawrence grades of knee Osteoarthritis. Ann Rheum Dis 2008; 67: 1034-6)

Follow up was performed within 6-12 months (Short-Term) and 24-48 months (Long-Term) with WOMAC, Harris Hip score and/or Knee Society score Questionnaires, Kellgren and Lawrence (K&L) Scale for Radiological Assessment.

Data were expressed as mean±SD and statistical analysis was performed through Student's t test. Differences were considered significant at the level of p<0.01. Statistical analysis was performed by using GraphPad Instat software (GraphPad Software, Inc; San Diego, Calif., USA).

The first group (I) consisted of 86 patients of which 49 patients were female and 37 male. The average age was 83 (range 80-91) (Table 1). The total number of joints treated was 123 of those (63 knees and 60 hips). 74 patients were followed-up, ranging from 6 to 48 months, 43 arrived at long-term (24-48 months) cut off. 12 patients were lost during the follow up (Table 2). The total number of therapeutic procedures performed on the 86 patients were 943 and percentage of response to therapeutic procedure was 92%. 13 patients were treated at different times on the same joint and 33 patients were treated at more than one joint. In this last case, the results had been analyzed separately. 34 patients underwent physiotherapy.

The second group (II) consisted of 90 patients of which 51 were female and 39 male. The average age was 51 (range 45-55) (Table 1). The total number of joints treated was 93 of those (44 knees and 49 hips). 80 patients were followed-up, ranging from 6 to 48 months, 52 arrived at long-term (24-48 months) cut off. 10 patients were lost during the follow up (Table 2). The total number of therapeutic procedures performed on the 90 patients were 800 and percentage of response to therapeutic procedure was 91%. 11 patients were treated at different times on the same joint and 3 patients were treated at more than one joint. In this last case, the results had been analyzed separately.

63 patients underwent physiotherapy. 21 patients underwent knee arthroscopy before being treated. 8 patients had been performed prosthesis during or next the follow-up was concluded.

As complication we report only wound subcutaneous infections on 1.2% of all patients.

The results of the questionnaires have been expressed as mean values for basic, short and long-term assessments as shown in (Table 3). The results of the radiological assessments expressed in Kellgren and Lawrence scale for basic, short and long-term follow-up is reported in Table 4; FIG. 1, 2, 3, 4, 5, 6.

Results of WOMAC questionnaire in both groups, respectively for the hip and the knee, show after treatment in the short term, a clear clinical improvement both in terms of perceived pain at rest and under stress, performance in terms of joint mobility and stability also improves. These results are confirmed in the long term (FIG. 7, 8).

The Harris Hip score and the Knee Society and Knee Society function score, that probe with an objective criterium clinical state of the patient, show substantial improvements coherent with WOMAC both in the short and the long-term for groups I and II (FIG. 9, 10, 11). In particular the Knee Society function score that evacuate the functional performance of the knee, we may notice that this parameter improves at the same rate as the clinical objective exam. (FIG. 11). Patients of both groups that had a radiological down staging from G IV to G III according to K&L scale, show in the follow-up, an average WOMAC score that is higher both as compared to the patients with down staging from G III to G II and, mainly, with the sub-group of patients unchanged for K&L staging. This evidence is statistically significant in group I between stages G IV/GIII and the sub-group of patients unchanged (FIG. 12).

TABLE 1
Patients' clinical features.

| Group I | | Group II | |
|---|---|---|---|
| Patients (n = 86); Lost* (n = 12) | | Patients (n = 90); Lost* (n = 10) | |
| Average age | 83 | Average age | 51 |
| (years) | | (years) | |
| Males/Females | 37/49 | Males/Females | 39/51 |
| Joint treated | Hip/knee | Joint treated | hip/knee |

Lost patients: they died during the treatment; they did not respond to the treatment; their follow up was missed

TABLE 2
Lost patients

| Group I | | Group II | |
|---|---|---|---|
| Patients (n = 86); Lost (n = 12) | | Patients (n = 90); Lost (n = 10) | |
| not responsive | 5 | not responsive | 8 |
| follow up lost | 5 | follow up lost | 2 |
| died during treatment | 2 | died during treatment | 0 |

TABLE 3
Average clinical baseline assessment and
classification before and after treatment.

| | | | |
|---|---|---|---|
| | average | average | average |
| | basic | short-term | long-term |
| Classifications | score^{a} | score^{b} | score^{c} |

Group I

| | | | |
|---|---|---|---|
| WOMAC | 31.3 | 75.2 | 73.7 |
| Harris Hip | 22.5 | 64.4 | 63.6 |
| Knee Society | 35.4 | 76 | 74.8 |
| Knee Society | 25.1 | 54.4 | 53.7 |
| Function | | | |

Group II

| | | | |
|---|---|---|---|
| WOMAC | 31 | 78.5 | 75.5 |
| Harris Hip | 22 | 67 | 65 |
| Knee Society | 36 | 76 | 75 |
| Knee Society | 25 | 60 | 58 |
| Function | | | |

^{a}basic score: before treatment.
^{b}short-term score: within 6-12 months of treatment.
^{c}long-termscore: within 24-48 months of treatment.

TABLE 4
Kellgren and Lawrence scale (K & L).

| | | |
|---|---|---|
| basic score^{a} | short-term score^{b} | long-term score^{c} |

Group I

| | | |
|---|---|---|
| 54 patients GIII | 7 patients GII | 4 patients GII |
| 32 patients GIV | 23 patients GIII | 14 patients GIII |

Group II

| | | |
|---|---|---|
| 50 patients GIII | 21 patients GII | 16 patients GII |
| 30 patients GIV | 20 patients GIII | 14 patients GIII |

^{a}basic score: before treatment.
^{b}short-term score: within 6-12 months of treatment.
^{c}long-term score: within 24-48 months of treatment.

The surgical pocket has be filled with the gel.

For this data two groups of patients were considered. A first group (I) consisted of over 80 years old who were judged high surgical risk and a second group (II) of young (45-55 years old) patients. Both of them were untreatable with the ordinary therapy. All patients were treated with local periarticular insertions of gel-repairer.

The data gathering started from questioners assessment which help to keep track of how the patient feels about his joint and how well he/she is able to perform his usual activity in terms of pain and function for everyday life. Statistical analysis of results shows a significant improvement of pain and joint motility (Table 3) and these improvements are similar in both groups and quite stable over time (FIGS. 7, 8, 9, 10, 11, 12).

Radiological modifications have been measured following Kellgren and Lawrence scale. In group (I) 30 out of 74 patients (41%) Kellgren and Lawrence scale has changed its grade towards a down-staging of the disease: 7 from grade III to II and 23 from IV to III (Tab 4). In group (II) 41 out of 80 patients (51%) have gotten a down-staging of DJD: 21 from grade III to II and 20 from IV to III (Tab 4). These results, though encouraging, only show part of the story. It is impossible to catch all the minute modifications that have great biological and clinical meaning through traditional radiological analysis, given that most of the impact occurs on the fibro-cartilaginous compartment and the capsular-ligament tissue. However patients who improved their K&L grade seemed to show the best clinical results during long-term follow-up. Comparing WOMAC and K&L scale results (FIG. 12) we can observe an evident correlation between structural and clinical improvements. On top of that, patients of both groups with greater anatomic improvement have more stable clinical results in the long term.

It would seem that results in terms of reduction of pain and improved articular functionality arise from a relatively stable improvement of the articular biomechanics induced by the gel on the treated areas. The hypothesis is that a prolonged action of proliferative and differentiative stimulus of polydeoxyribonucleotides, heat shock protein and of other growth factors on the cellular blastic periarticular compartment, together with the scaffold function that gel might have on the activated stem cells produces a structural change of the joint. This is confirmed by the improved articular flexibility that goes with the reduced pain. This flexibility seems to be caused by a tissue modification that translates into an increased compliance of the ligament bursa tissue, which reduces intra-articular pressure.

Traditional radiology shows an increase in thickness of the soft tissue layer in 30 out of 74 cases and in 51 out of 80 respectively in the first and in the second group. These patients, in turn, show better overall results in the long term. In other cases, MR shows that the intensity of the signal of the neo matrix is similar to that produced by the fibro-cartilaginous interarticular layer. Furthermore traditional radiology limits in highlighting the structural modifications of soft tissue become apparent when Rx and MR images of the same patient are compared. We believe that the greater specificity and sensitivity of MR will increase the percent of patients showing a down-staging, once standard criteria will be introduced (FIG. 13). Given the absence to date of a standard criterium of MR, this data could not be statistically sampled and used in this study but only showed as case report.

The histological interpretation of this radiological and structural joints improvement expressed as an increase in thickness of radiolucent layer between the head joints still has to be accounted for.

FIG. 13 shows Rx vs MR of 53 years old patient before and after treatment. In this comparative figures has shown the right knee of a man who underwent gel-repairer treatment mostly on medial joint compartment.

X-rays imaging do not change from 2005 to 2010 and so K&L scale is steady. Instead comparing MR imagery after and before treatment, is evident the recovery of thickness in the soft tissue layer and a great improvement over bone damage disappearing erosion and pseudo-cyst marrow.

1-13. (canceled)

14. Haematic component obtainable by the process comprising the following steps
a) subjecting blood or a blood derivative, said blood derivative comprising at least one cellular component, to thermal treatment at a temperature between −10 e-50° C. for at least 12-16 hours,
b) subjecting the product obtained in step a) to thermal treatment at a temperature between 100 and 200° C. for a time between 60 and 200 minutes.

15. Haematic component according to claim 14 wherein said blood is autologous blood.

16. Composition comprising the haematic component of claim 14 and a fluidifying agent.

17. Composition according to claim 16 further comprising PDRN.

18. Composition according to claim 16 further comprising at least one heat shock protein.

19. Composition according to claim 16 further comprising mesenchimal stem cells.

20. Composition comprising the haematic component of claim 14 and a thickener.

21. Composition according to claim 20 further comprising PDRN.

22. Composition according to claim 20 further comprising at least one heat shock protein.

23. Composition according to claim 22 characterised in that it is in the form of a gel.

24. A composition comprising the haematic component of claim 15 and a fluidifying agent.

25. A composition comprising the haematic component of claim 15 and a thickener.

26. A method of treating not-autoimmune degenerative joint disease, comprising administering to a subject in need thereof an effective amount of the haematic component of claim 14.

27. A method of treating not-autoimmune degenerative joint disease, comprising administering to a subject in need thereof an effective amount of the haematic component of claim 15.

28. A method of treating not-autoimmune degenerative joint disease, comprising administering to a subject in need thereof an effective amount of the composition of claim 16.

29. A method of treating not-autoimmune degenerative joint disease, comprising administering to a subject in need thereof an effective amount of the composition of claim 20.

30. A kit comprising the haematic component of claim 14 and means for intra-articular and peri-articular administration.

31. A kit comprising the composition of claim 16 and means for intra-articular and peri-articular administration.

32. A kit comprising the composition of claim 20 and means for intra-articular and peri-articular administration.
